SEQUENCE LISTING (17 sequences)

SEQ ID NO: 1 - DNA, 903 bp, Escherichia coli
  CDS (1)..(903)
atgaaacgagttattaccctgtttgctgtactgctgatgggctggtcggtaaatgcctggtcattcgcctgtaaaaccgccaatggtaccgcaatccctattggcggtggcagcgccaatgtttatgtaaaccttgcgcctgccgtgaatgtggggcaaaacctggtcgtagatctttcgacgcaaatcttttgccataacgattacccagaaaccattacagactatgtcacactgcaacgaggtgcggcttatggcggcgtgttatctagtttttccgggaccgtaaaatataatggcagtagctatcctttccctactaccagcgaaacgccgcgggttgtttataattcgagaacggataagccgtggccggtggcgctttatttgacgccggtgagcagtgcggggggagtggcgattaaagctggctcattaattgccgtgcttattttgcgacagaccaacaactataacagcgatgatttccagtttgtgtggaatatttacgccaataatgatgtggtggtgcccactggcggctgcgatgtttctgctcgtgatgtcaccgttactctgccggactaccctggttcagtgccgattcctcttaccgtttattgtgcgaaaagccaaaacctggggtattacctctccggcacaaccgcagatgcgggcaactcgattttcaccaataccgcgtcgttttcacccgcgcagggcgtcggcgtacagttgacgcgcaacggtacgattattccagcgaataacacggtatcgttaggagcagtagggacttcggcggtaagtctgggattaacggcaaattacgcacgtaccggagggcaggtgactgcagggaatgtgcaatcgattattggcgtgacttttgtttatcaataa

SEQ ID NO: 2 - protein, 300 aa, Escherichia coli
MKRVITLFAVLLMGWSVNAWSFACKTANGTAIPIGGGSANVYVNLAPAVNVGQNLVVDLSTQIFCHNDYPETITDYVTLQRGAAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 3 - DNA, 3738 bp, Artificial Sequence
  Recombinant pSTM27 bacteriophage
gcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaagatctagctattctagagattacgccaagccccgtattttacccgtttaatggaaacttcctcatgaaaaagtctttagtcctcaaagcctctgtagccgttgctaccctcgttccgatgctgtctttcgctgctgagggtgaattccataacgattatccggaaaccattacagactatgtcacactgcaacgaggctcggcttatggcggcgtgttatctaatttttccgggaccgtaaaatatagtggcagtagctatccatttcctaccaccagcgaaacgccgcgcgttgtttataattcgagaacggataagccgtggccggtggcgctttatttgacgcctgtgagcagtgcgggcggggtggcgattaaagctggctcattaattgccgtgcttattttgcgacagaccaacaactataacagcgatgatttccagtttgtgtggaatatttacgccaataatgatgtggtggtgcccactggagactacaaagacgacgacgacaaatcaggtggaggcggttcaggcggaggtggctctggcggtggcggatcggatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagctaagcggccgcaggagactacaaagacgacgacgacaaacaccaccatcaccaccattaaggccgctaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 4 - protein, 213 aa, Artificial Sequence
  Synthetic Construct
MKKSLVLKASVAVATLVPMLSFAAEGEFHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGDYKDDDDKSGGGGSGGGGSGGGGSDPAKAAFDSLQASATEYIGYAWAMVVVIVGATIGIKLFKKFTSKAS

SEQ ID NO: 5 - DNA, 222 bp, Bacteriophage M13mp18
  CDS (1)..(222)
atgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctga

SEQ ID NO: 6 - protein, 73 aa, Bacteriophage M13mp18
MKKSLVLKASVAVATLVPMLSFAAEGDDPAKAAFNSLQASATEYIGYAWAMVVVIVGATIGIKLFKKFTSKAS

SEQ ID NO: 7 - DNA, 228 bp, Artificial Sequence
  pc89 major coat protein PVIII
atgaaaaagtctttagtcctcaaagcctctgtagccgttgctaccctcgttccgatgctgtctttcgctgctgagggtgaattctaggatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagctga

SEQ ID NO: 8 - protein, 74 aa, Artificial Sequence
  Synthetic Construct
MKKSLVLKASVAVATLVPMLSFAAEGEFDPAKAAFDSLQASATEYIGYAWAMVVVIVGATIGIKLFKKFTSKAS

SEQ ID NO: 9 - DNA, 41 bp, Artificial Sequence
  synthetic primer
ctgctgctgccatggtttgtaaaaccgccaatggtaccgct

SEQ ID NO: 10 - DNA, 40 bp, Artificial Sequence
  synthetic primer
gtcgtctgcggccgcagtgggcaccaccacatcattattg

SEQ ID NO: 11 - DNA, 39 bp, Artificial Sequence
  synthetic primer
ctgctgctggaattctgtaaaaccgccaatggtaccgct

SEQ ID NO: 12 - DNA, 113 bp, Artificial Sequence
  synthetic primer
ctgctgctggatccgatccgccaccgccagagccacctccgcctgaaccgcctccacctgatttgtcgtcgtcgtctttgtagtctccagtgggcaccaccacatcattattg

SEQ ID NO: 13 - DNA, 41 bp, Artificial Sequence
  synthetic primer
ctgctgctggaattccataacgattatccggaaaccattac

SEQ ID NO: 14 - protein, 8 aa, Artificial Sequence
  FLAG-tag
DYKDDDDK

SEQ ID NO: 15 - protein, 16 aa, Artificial Sequence
  peptide linker
SGGGGSGGGGSGGGGS

SEQ ID NO: 16 - DNA, 20 bp, Artificial Sequence
  CpG oligonucleotide
tccatgacgttcctgacgtt

SEQ ID NO: 17 - DNA, 20 bp, Artificial Sequence
  non CpG oligonucleotide
tccatgagcttcctgacgtt

(19) United States
(12) Patent Application Publication
(10) Pub. No.: US 2013/0122033 A1
(43) Pub. Date: May 16, 2013

(54) FIMH VACCINE AGAINST URINARY TRACT INFECTIONS (UTI)

(71) Applicants: Rita De Santis, Pomezia (RM) (IT); Olga Minenkova, Montecompatri (RM) (IT)

(72) Inventors: Rita De Santis, Pomezia (RM) (IT); Olga Minenkova, Montecompatri (RM) (IT)

(21) Appl. No.: 13/806,609

(22) PCT Filed: Jun. 28, 2011
(86) PCT No.: PCT/EP2011/060839

§ 371 Date: Jan. 30, 2013

(30) Foreign Application Priority Data
Jul. 2, 2010 (EP) 10168280.5

(51) Int. Cl. A61K 39/02

(52) U.S. Cl. CPC A61K 39/02
USPC 424/190.1; 435/235.1

(57) ABSTRACT

A engineered bacteriophage is herein described, displaying at its surface multiple copies of a polypeptide, which comprises a domain of the bacterial adhesion protein FimH fused with the coat protein pVIII. The domain is preferably the region 45-159aa of the mature protein FimH. The polypeptide beingantigenic, raises a desired immunological response, in particular against urinary tract infections (UTI) in a human or animal. The engineered bacteriophage is therefore suitable for the preparation of vaccinesfor therapy, diagnosis and/or prevention of urinary tract infections (UTI) in a human or animal.

### FIELD OF THE INVENTION

The present invention relates to vaccines comprising a bacteriophage, which has been engineered to display at its surface an exogenous polypeptide as fusion with the coat protein, i.e. pVIII. Such exogenous polypeptide is in particular a domain of the bacterial adhesion protein FimH.

### BACKGROUND OF THE INVENTION

Urinary-tract infections (UTIs) are among the more wide-spread bacterial diseases that occur in humans (Hooton et al., 1997). Main causative agent in these infections is uropathogenic E. coli (UPEC) (Stamm, 2004). Recent studies demonstrated that recurrent infections may originate not only from fecal and vaginal flora but also from intracellular bacterial communities (IBC) within epithelial cells of the bladder (Mysorekar et al., 2006; Garofalo et al., 2007; Reigstad et al., 2207). Biofilm formation by IBC allows the bacteria to persist a host immune response and an antibiotic treatment.

Vaccine available for recurrent UTIs are formulated with whole inactivated bacterial cells and administered orally (Schulman et al., 1993) or intravaginally (Pam, 2002; Hopkins et al., 2007). This type of vaccine has a limited success (Naber et al., 2008) and it is supposed to be improved. Inactivated vaccines Uro-Vaxom® (OM-Pharma©) and Solco-Urovac® (Solco Basle©) are not approved by FDA (Food and Drug Administration). In 1997, Langermann showed that recombinant vaccine based on an adhesin bacterial protein FimH efficiently blocks infection in murine model of disease (Langetmann S et al., 1997). Use of the recombinant FimH is also an efficient method to induce a high level IgA by intranasal vaccination when it is using with synthetic oligonucleotides containing CpG as an adjuvant (Poggio T V et al., 2006).

Authors developed the filamentous phage-based vaccine displaying mannose-binding lectin domain of adhesin protein FimH. Good immunogenic properties of filamentous phage displaying short peptides are well-known (Minenkova O O et al., 1993; Willis A E et al., 1993; Lidgvist M et al., 2008; Esposito M et al., 2008). Moreover, filamentous phage displaying numerous copies of antigenic polypeptide are able to elicit an immune response without adding particular adjuvant because of polymeric virus-like phage particle structure (Willis A E et al., 1993).

Bacteriophages replicate solely in bacterial cells and have no potential host cells in the human organism (Clark et al., 2004). Safety of phage vaccination can be confirmed by long-time application of one coliphage, φX174, for monitoring of both primary and secondary immunodeficiency diseases (Ochs et al., 1971; Wedgwood et al., 1975; Bernstein et al., 1985). In fact, over 30 years φX174 is considered to be one of the standard antigens for the evaluation of humoral immunity in clinical medicine (Bearden et al., 2005).

### DESCRIPTION OF THE INVENTION

In the present application authors show that FimH domain was efficiently displayed on the surface of the filamentous phage as fusion with major coat protein pVIII (FIG. 10). Preferably, to improve the efficiency of the display and to increase the yield of the recombinant phage authors cloned a FimH domain of 114 aa, obtaining an embodiment of the phage of the invention, named pSTM27. The FimH domain still contained nearly all amino acid residues forming the binding site to adhere to the bladder epithelial cell. Authors found the pSTM27 forms phage particles stable for a long storage (more than 1 year at 4° C.) and resists to purification on the CsCl gradient without any degradation of the displayed polypeptide or reduction of the specific reactivity with anti-FimH sera.

The obtained phage pSTM27 was used for mice immunization. The rodents were vaccinated with pSTM27 phage by using intranasal and intramuscular routes of immunization. The intranasal vaccination was performed by using the bacteriophage alone, intramuscular did in combination with Freund's adjuvant, CpG oligonucleotide and control non-CpG oligonucleotide.

Results showed high serum levels of anti-FimH IgG in animals vaccinated i.m. and the presence of specific IgA against FimH protein in vaginal-wash of mice after both intramuscular and intranasal immunizations. Moreover, recombinant vaccines reduced 10 times in vivo colonization of the bladder with clinical UPEC strain, i.e. from laboratory collection PT27 in mice immunized intranasally.

Moreover bacteriophage-based vaccine would have several important advantages:

1. inexpensive production,
2. growth media are free of animal-derived materials,
3. phage preparations are stable at 4° C. for a long time,
4. phage is biologically different from human viruses, can not infect mammals,
5. phage physical structure is similar to virus, they work as adjuvant and responses to the vaccine component are increased,
6. polyclonal vaccines can be easily prepared.

Object of the invention is therefore a recombinant bacteriophage displaying at its surface multiple copies of a chimeric polypeptide, said chimeric polypeptide comprising at least:

a) a coat protein of a filamentous bacteriophage or a functional fragment thereof able to be expressed on the surface of the bacteriophage and
b) an immunogenic domain of the bacterial adhesion protein FimH consisting of SEQ ID No. 2, wherein the filamentous bacteriophage preferably belongs to the group of M13, fd and fl.

The coat protein is preferably the coat protein pVIII.

Preferably, the immunogenic domain of the bacterial adhesion protein FimH is a lectin-binding domain, more preferably said domain is the fragment 24-179 aa of SEQ ID No. 2 or the fragment 66-180 aa of SEQ ID No. 2.

Said chimeric polypeptide preferably comprises a linker between the coat protein or fragment thereof and the immunogenic domain of the bacterial adhesion protein FimH.

The above chimeric polypeptide preferably comprises an affinity tag.

Preferably, the recombinant bacteriophage of the invention has the nucleic acid sequence essentially consisting in SEQ ID No. 3.

One object of the present invention is therefore the newly engineered bacteriophage of the invention, which includes a foreign polypeptide: the polypeptide should be antigenic, e.g. any polypeptide that raises a desired immunological response, in particular against UTI pathogens, as Uropathogenic E. coli (UPEC). Its length should be sufficient to raise the response but insufficient to modify the bacteriophage's properties undesirably or to prevent its incorporation. A bacteriophage of the invention is preferably immunogenic, and is suitable for use in vaccines, and generally as a therapeutic/diagnostic product. Therefore, products of the invention may be formulated with any suitable physiologically-acceptable diluent or carrier, to prepare a vaccine composition.

The hybrid phage of the invention preferably allows the number of copies of the exogenous polypeptide displayed on each viral particle to be controlled within wide limits, and can confer great sensitivity.

As it is known, the major coat protein of the filamentous bacteriophages, preferably M13, fd, fl, is encoded by gene VIII. The protein is synthesized as a procoat which contains a 23-amino acid leader peptide attached to the N-terminus of the mature protein. After synthesis, the procoat protein is rapidly inserted into the inner-membrane of E. coli where it is processed to leave the 50-amino acid mature coat protein spanning the membrane. This protein has three domains, a hydrophobic membrane-spanning domain, a positively-charged C-terminal domain which faces into the cytoplasm of the cell, and a negatively-charged N-terminal domain which extends into the periplasm. During assembly of virus particles, the coat protein subunits are pulled out of the cell membrane and become arranged in a helical array around the viral DNA. In the viral particle, the C-terminal region of the protein subunits faces inwards towards the DNA such that the positively-charged residues may neutralize the negative charges of the sugar-phosphate backbone. The N-terminal domain, by contrast, is on the outer surface of the particle where it is exposed to the environment. Nuclear magnetic resonance studies have indicated that this region is flexible both in the membrane-bound form of the protein and when presented on the outside of the phage particle. In the invention, the exogenous polypeptide is fused to the amino terminus of a phage major coat protein, to give extended coat proteins that successfully assemble into viral particles and elicit a significant immune response. This attachment can be direct or through one or more suitable linkers.

Therefore, according to a preferred embodiment of the invention, the selected FimH domain is inserted into the N-terminal domain of the major coat protein of filamentous bacteriophage M13 (pVIII) by using (SGGGG)₃S (SEQ ID No. 15) flexible linker, such that they “decorate” the outside of an assembled phage particle.

The preparation of this phage may be approached entirely at the DNA level, by engineering a recombinant phage using restriction enzyme recognition sites in gene VIII, and allowing insertion of a foreign DNA fragment, encoding for the desired polypeptide, directly into the phage genome. In order to achieve this result in a controlled manner, however, it may be preferred to separate the synthesis of any hybrid coat proteins from the assembly of phage particles. This allows the cloning and expression of novel coat protein constructs, and the membrane-insertion and packaging stages of phage assembly, to be carried out as independent steps.

In order to allow independent expression of the M13 coat protein gene, gene VIII may be subcloned into a controllable expression vector, phagemid. However, the wild-type gene VIII does not contain a suitable restriction enzyme site for the insertion of oligonucleotide cassettes (which would be used to encode the epitopes). Therefore, before the gene is subcloned, a suitable site may be introduced by means of site-directed mutagenesis.

In preparing a hybrid bacteriophage of the invention, an intermediate product is a plasmid encoding the modification. In order to prevent the loss of such a plasmid, it is preferred that the engineered coat protein is transplanted into a phage, phagemid or other replicon, with a packaging signal. The recombinant gene can then be packaged and selected for further replication.

Preferably the engineered phage of the invention is a filamentous bacteriophage, more preferably is M13 or related phagemid.

M13 is a filamentous bacteriophage (GenBank V00604) composed of circular single stranded DNA (ssDNA) which is 6407 nucleotides long encapsulated in approximately 2700 copies of the major coat protein pVIII, and capped with 5 copies of four different minor coat proteins (pIX, pVII, pVI, pIII) on the ends. The minor coat protein pIII attaches to the receptor at the tip of the F pilus of the host Escherichia coli. Infection with filamentous phages is not lethal; however the infection causes turbid plaques in E. coli. It is a non-lytic virus. However a decrease in the rate of cell growth is seen in the infected cells. M13 plasmids are used for many recombinant DNA processes, and the virus has also been studied for its uses in nanostructures and nanotechnology.

The phage coat is primarily assembled from a 50 amino acid protein called pVIII (or P8), which is encoded by gene VIII (or g8) in the phage genome. For a wild type M13 particle, it takes about approximately 2700 copies of pVIII to make the coat shell about 900 nm long. The coat's dimensions are flexible though and the number of pVIII copies adjusts to accommodate the size of the single stranded genome it packages. For example, when the phage genome was mutated to reduce its number of DNA bases (from 6.4 kb to 221 bp), then the number of pVIII copies was decreased to fewer than 100, causing the pVIII coat to shrink in order to fit the reduced genome. The phage appear to be limited at approximately twice the natural DNA content. However, deletion of a phage protein (pIII) prevents full escape from the host E. coli, and phage that are 10-20× the normal length with several copies of the phage genome can be seen shedding from the E. coli host.

A further object of the inventions is the recombinant bacteriophage of the invention for use as a medicament, in particular for use as an immunogen against urinary tract infections (UTI).

Another object of the invention is a pharmaceutical composition comprising the recombinant bacteriophage of the inventions, in association with a physiologically-acceptable carrier or diluent, said pharmaceutical composition being preferably capable of eliciting a humoral and/or cellular immune response.

A further object of the inventions is said pharmaceutical composition for use in vaccinating against urinary tract infections (UTI).

The above pharmaceutical composition can further comprise an adjuvant.

The recombinant bacteriophage is preferably associated with a vehicle in the pharmaceutical composition.

Another object of the invention is the pharmaceutical composition of the invention for use in the prophylaxis and or treatment of urinary tract infections (UTI) in a human or animal.

In an important aspect the present invention provides a vaccine formulation comprising the bacteriophage particle as explained before, comprising the exogenous nucleic acid molecule encoding a polypeptide which is capable of expression and presentation on the surface of an antigen presenting cell of an organism, such that an immune response to said polypeptide is raised in the organism.

The bacteriophage of the present invention is thought to be recognised as “foreign” and therefore processed in the normal manner by a host's immune system. Moreover, by modifying the genome of the bacteriophage to include exogenous nucleic acid capable of encoding a foreign polypeptide/protein, that is a polypeptide/protein not normally present in a chosen mammalian host, an immune response to this foreign protein is elicited. It is to be appreciated that the immune response may be a humoral (ie. antibody) and/or cellular immune response.

The term “exogenous” relates to any material (e.g. polypeptide or nucleic acid) that is present and active in an individual organism or biological entity (e.g. bacteriophage), but that in nature is originated outside of that organism, as opposed to an endogenous factor.

The exogenous polypeptide or protein is expressed at a level sufficient to elicit an immune response in a host to which the vaccine has been presented.

It is to be appreciated that the present invention is applicable to the preparation of a vaccine for UTI.

The exogenous polypeptide on the surface of the bacteriophage allows direct uptake of nucleic acid specifically to APC. Without being bound by theory it is expected the bacteriophage particle is recognised as a “foreign” antigen. In general the term “polypeptide” here refers to a chain or sequence of amino acids displaying an antigenic activity and does not refer to a specific length of the product as such. The polypeptide if required, can be modified in vivo and/or in vitro, for example by glycosylation, amidation, carboxylation, phosphorylation and/or post translational cleavage, thus inter alia, peptides, oligo-peptides, proteins and fusion proteins are encompassed thereby.

Naturally the skilled addressee will appreciate that a modified polypeptide should retain physiological function i.e. be capable of eliciting an immune response.

The bacteriophage of the present invention preferably contain appropriate transcription/translation regulators such as promoters, terminators and/or the like.

Conveniently the promoter may be a constitutive promoter.

However, controllable promoters known to those of skill in the art may also be used. For example constructs may be designed which comprise the exogenous nucleic acid under control of a constitutive promoter and a controllable promoter. In this manner it may be possible to cause expression of the exogenous nucleic acid initially by way of the constitutive promoter and at a second time point by expression from the controllable promoter. This may result in synthesis of more immunogenic phage particles.

In a preferred embodiment of the invention the phage could be modified to express the antigenic protein on the surface of the phage particle. For example it is possible to use intact bacteriophage M13 particles as a vector vehicle. Insert sizes for M13 are relatively small, but the use of “Phage Display” technology (Hawkins, R E et al. 1992, J. Mol. Biol. 226: 889) means that the phage particle can carry a portion of foreign antigen fused to its coat protein. Thus a construct can be made in which the exogenous gene is under control of a prokaryotic (eg. LacZ promoter) promoter: when grown in an E. coli host, the prokaryotic promoter will direct expression of the vaccine antigen and allow its incorporation into the M13 coat as a fusion protein, which should elicit a strong primary response following vaccination.

Preferably the exogenous nucleic acid according to the present invention comprises or consists of the DNA coding for the mannose-binding lectin domain of FimH, more preferably the domain coding for 45-159 aa of the mature protein FimH (here also named ΔFimH).

More preferably the exogenous nucleic acid may comprise a sequence coding for affinity tags. Examples of such affinity tags are FLAG-tag, or FLAG octapeptide, polyhistidine tag (His-tag), HA-tag or myc-tag. FLAG-tag is a polypeptide protein tag that can be added to a protein using recombinant DNA technology, in order to purify recombinant bacteriophage using affinity chromatography or for quality control of the fusion protein expression. A preferred peptide sequence of the FLAG-tag is as follows: N-DYKDDDDK-C (SEQ ID No. 14) (1012 Da). It can be used in conjunction with other affinity tags. It can be fused to the C-terminus or the N-terminus of a protein. Moreover the construction of a fusion protein usually involves the linking of two proteins or domains of proteins by a peptide linker. The selection of the linker sequence is particularly important for the construction of functional fusion proteins. The flexibility and hydrophilicity of the linker are generally important not to disturb the functions of the domains, in our case the exogenous polypeptide and the protein coat pVIII. A preferred linker which has been successfully used in the literature to engineer recombinant antibodies displayed on bacteriophages is (SGGGG)₃S (SEQ ID No. 15).

A specific object of the invention is therefore the nucleic acid sequence of the bacteriophage engineered according to the present invention, as for example shown in FIG. 9, as well as all nucleotide sequences, which are substantially the same. “Nucleotide sequences substantially the same” includes all other nucleic acid sequences that, by virtue of the degeneracy of the genetic code, also code for the given amino acid sequences.

The bacteriophage may be administered by any suitable route, for example by injection and may be prepared in unit dosage form in for example ampules, or in multidose containers. The bacteriophage may be present in such forms as suspensions, solutions, or emulsions in oily or preferably aqueous vehicles. Alternatively, the bacteriophage may be in lyophilized form for reconstitution, at the time of delivery, with a suitable vehicle, such as sterile pyrogen-free water. In this manner stabilising agents, such as proteins, sugars etc. may be added when lyophilising the phage particles. Both liquid as well as lyophilized forms that are to be reconstituted will comprise agents, preferably buffers, in amounts necessary to suitably adjust the pH of the injected solution. For any parenteral use, particularly if the formulation is to be administered intravenously, the total concentration of solutes should be controlled to make the preparation isotonic, hypotonic, or weakly hypertonic.

Nonionic materials, such as sugars, are preferred for adjusting tonicity, and sucrose is particularly preferred.

Any of these forms may further comprise suitable formulatory agents, such as starch or sugar, glycerol or saline. The compositions per unit dosage, whether liquid or solid, may contain from 0.1% to 99% of bacteriophage material.

In a preferred presentation, the vaccine can also comprise an adjuvant. Adjuvants in general comprise substances that boost the immune response of the host in a non-specific manner. A number of different adjuvants are known in the art. Examples of adjuvants may include Freund's Complete adjuvant, Freund's Incomplete adjuvant, liposomes, and niosomes as described, for example, in WO 90/11092, mineral and non-mineral oil-based water-in-oil emulsion adjuvants, cytokines, short immunostimulatory polynucleotide sequences, for example in plasmid DNA containing CpG dinucleotides such as those described by Sato Y. et al. (1996) Science Vol. 273 pp. 352-354; Krieg A. M. (1996) Trends in Microbiol. 4 pp. 73-77.

The bacteriophage may also be associated with a so-called “vehicle”. A vehicle is a compound, or substrate to which the bacteriophage can adhere, without being covalently bound thereto. Typical “vehicle” compounds include gold particles, silica particles such as glass and the like. Thus the bacteriophage of the invention may be introduced into an organism using biolistic methods such as the high-velocity bombardment method using coated gold particles as described in the art (Williams R. S. et al. (1991) Proc. Natl. Acad. Sci. USA 88 pp. 2726-2730; Fynan E. F. et al. (1993) Proc. Natl. Acad. Sci. USA Vol. 90 pp. 11478-11482).

In addition, the vaccine may comprise one or more suitable surface-active compounds or emulsifiers, eg. Span or Tween.

The mode of administration of the vaccine of the invention may be by any suitable route which delivers an immunoprotective amount of the virus of the invention to the subject. However, the vaccine is preferably administered parenterally via the intramuscular or deep subcutaneous routes. Other modes of administration may also be employed, where desired, such as via mucosal routes (eg. rectal, oral, nasal or vaginal administration) or via other parenteral routes, ie., intradermally, intranasally, or intravenously. Formulations for nasal administration may be developed and may comprise for example chitosan as an adjuvant (Nat. Medicine 5 (4) 387-92, 1999).

It will be understood, however, that the specific dose level for any particular recipient organism will depend upon a variety of factors including age, general health, and sex; the time of administration; the route of administration; synergistic effects with any other drugs being administered; and the degree of protection being sought. Of course, the administration can be repeated at suitable intervals if necessary. Usually a daily dosage of vaccine is comprised between 0.01 to 100 milligrams per kilogram of body weight.

In a further aspect therefore, the present invention provides a method of immunising, prophylactically and/or therapeutically, a human or animal, comprising administering to the human and/or animal an effective dose of a vaccine formulation as described herein. It being understood that an effective dose is one which is capable of eliciting an immune response in the human and/or animal.

All references cited herein are entirely incorporated by reference herein, including all data, tables, figures, and text presented in the cited references. Additionally, the entire contents of the references cited within the references cited herein are also entirely incorporated by reference. Reference to known method steps, conventional method steps, known methods or conventional methods is not in any way an admission that any aspect, description or embodiment of the present invention is disclosed, taught or suggested in the relevant art.

Once understood the features of the methods and products disclosed in present application, the necessity and kind of additional steps can be easily deduced by reviewing prior art, as well as the non-limiting following figures and examples describing the basic details and some applications of the invention

The present invention will now be further described by way of Example and with reference to the following Figures.

### DESCRIPTION OF THE FIGURES

FIG. 1 shows the phage reactivity against anti-FLAG antibody. ELISA plate was coated with anti-pIII monoclonal antibody and developed after phage binding with an anti-FLAG HRP-conjugated secondary antibody. Wild type phage M13KO7 (Vieira & Messing, 1987) was included as a negative control. The pSTM23 displays FimH protein fused with pVIII protein, tagged with the FLAG peptide.

FIG. 2 shows phage reactivity against anti-FimH serum. Anti-FimH serum specifically recognizes the pSTN23 phage, displaying FimH domain on the surface of the phage particle. The plate was coated with anti-pIII monoclonal antibody and after phage binding was developed with different dilutions of anti-FimH serum and an anti-rabbit HRP-conjugated secondary antibody.

FIG. 3 reports the nucleotide (SEQ ID No. 1) and amino acid (SEQ ID No. 2) sequence of bacterial FimH adhesin protein (GenBank NC 007946). The amino sequence of FimH protein is shown: the amino sequence of the leader peptide is shown in bold (1-21 aa), the amino acid sequence of the mannose-binding lectin domain is shown underlined (22-179 aa). The phage pSTM23 displays the fragment 24-179 aa, corresponding to 3-158 aa of the mature protein, of FimH, shown in bold and underlined, while the pSTM27 displays the domain 66-180 aa, corresponding to 45-159 aa of the mature protein, of FimH, here named ΔFimH, shown in bold and italic. Cysteine residues in the mannose-binding lectin domain are marked in a larger font; the amino acid residues participating in mannose binding (according to Tchesnokova et al., 2008) are marked with (*).

FIG. 4 shows the competition with FimH protein. The plate was coated with anti-pIII monoclonal antibody. About 3×10⁹ CFU of phage was added to the wells. Anti-FimH serum was added to the well in dilution 1:100 alone or in combination with 5 mg of FimH protein or irrelevant protein GST and developed at the end with an anti-rabbit HRP-conjugated secondary antibody. FimH protein efficiently competes with the phages displaying lectin domain for binding to anti-FimH serum.

FIG. 5 reports the analysis of reactivity pSTM27 phage purified in CsCl gradient. The pSTM27 phage concentrated by PEG/NaCl precipitation was analyzed in comparison with the phage purified additionally on CsCl gradient. The plate was coated with anti-pIII monoclonal antibody and developed at the end with anti-FimH serum diluted 1:50 and an anti-rabbit HRP-conjugated secondary antibody.

FIG. 6 shows the average anti-FimH immunoglobulin G serum titer in the groups of mice immunized with bacteriophage pSTM27. Serum samples from individual mice were obtained just before bacterial challenge, on the 45^{th} day after vaccination. Individual antibody titers were calculated as highest serum dilution factor resulting in an absorbance value which is still higher than twice the value for unimmunized animals The bars on the figure show mean values of antibody titers for immunization groups.

FIG. 7 shows anti-FimH immunoglobulin A specific antibody levels in vaginal washes in the mice immunized with bacteriophage pSTM27. Vaginal washes from individual mice were obtained just before bacterial challenge, on the 45^{th} day after vaccination.

FIG. 8 shows the results of in vivo bacterial challenge with uropathogenic E. coli PT27 (UPEC) in mice vaccinated with bacteriophage pSTM27. All mice were challenged on day 45 with 10⁸ CFU of UPEC strain PT27.

FIG. 9 reports the nucleotide sequence of the pSTM27 (SEQ ID No. 3). The amino acid sequence of the fused pVIII-FimH protein (SEQ ID No. 4) is shown: the amino acid of the leader peptide of the fused pVIII protein and the starting ATG codon are shown in bold; the amino acid sequence of the pVIII is shown underlined; the amino acid sequence of the FimH is shown in italic; the amino acid sequence of the FLAG peptide is shown in bold and underlined; the amino acid sequence of the flexible linker (SGGGG)₃S (SEQ ID No. 15) is shown in bold and italic.

FIG. 10. (A) Phage M13 major coat protein pVIII (GenBank: V00604.2). The nucleotide (SEQ ID No. 5) and amino acid (SEQ ID No. 6) sequences of the major coat protein (pVIII) of the filamentous phage M13. The leader peptide of pVIII protein is marked in bold.

(B) pc89 major coat protein pVIII (Felici et al., 1991). The nucleotide (SEQ ID No. 7) and amino acid (SEQ ID No. 8) sequences of the major coat protein (pVIII) encoded in pc89 (Felici et al., 1991). The leader peptide of pVIII protein is marked in bold, cloning sites EcoRI and BamHI are shown in bold and underlined, amber codon is shown in italic.

### EXAMPLES

### Materials and Methods

### Bacterial Strains

A clinic isolate of UPEC PT27 was chosen to infect animals in the murine model of cystitis. This strain was originally obtained from Laboratory of Clinic Analysis DiFi (Pomezia (RM), Italy) from the urine of a patient with recurrent UTI. The PT27 strain was tested for type 1 pili expression, using a mannose-sensitive hemagglutination assay and an inhibition of hemagglutination by mannose.

Laboratory E. coli strain DH5αF′ (supE44 ΔlacU169 (φ80 lacZΔM15) hsdR17 recA1 endA1 rA96 thi-1 relA1 F′ [traD36 proAB⁺ lacI^{q}lacZΔM15]) was used for phage propagation.

### Cloning and Expression FimH Lectin Domain

The DNA fragment encoding for FimH lectin domain was amplified from DH5αF′ genome with primers SM124 FOR (5′-CT GCT GCT GCC ATG GTT TGT AAA ACC GCC AAT GGT ACC GCT-3′(SEQ ID No. 9)) and SM125 REV (5′-GTC GTC TGC GGC CGC AGT GGG CAC CAC CAC ATC ATT ATT G-3′(SEQ ID No. 10)) containing NcoI and NotI sites respectively (underlined). The resulting PCR product was gel purified, digested with NcoI and Nod, and ligated into the plasmid of pKM16 (Pavoni et al., 2007), in turn also digested with NcoI and Nod. The resulting plasmid pSTM21 was used for FimH expression.

### Production of Anti-FimH Polyclonal Serum in Rabbit

The FimH domain was expressed in E. coli DH5αF′ and purified according to following protocol. Bacterial cells transformed with pSTM21 were cultured with agitation ON in LB containing 100 μg/mL Ap. Next morning one ml of the ON culture was inoculated in 100 mL of the fresh medium. The culture were grown up to OD=0.8 and the FimH expression was induced by adding 0.1 mM IPTG. After ON incubation at 37° C. the cells were harvested, resuspended in 10 mL PBS containing 1 mg/mL lysozyme, incubated 30 min on the ice in the presence of protease inhibitor (Roche). After adding 1.5% N-laurylsarcosine to increase protein solubility the cells suspension was homogenized by sonicating. The cell debris was span down and FimH protein expressed with His-tail was purified by using His-Select HF Nickel Affinity Gel (Sigma) according to manufacturer's instructions. The purified protein was analyzed by SDS-PAGE on a 12% gel and then it was transferred onto a nitrocellulose membrane and immunostained by an anti-FLAG secondary antibody.

The purified FimH protein was used for vaccination of rabbits to obtain polyclonal rabbit anti-FimH serum.

### Display of FimH and ΔFimH Domain on Filamentous Phage

The DNA fragment coding for the FimH lectin domain (3-158 aa of the mature protein) was amplified from DH5αF′ genome with primers SM126 FOR (5′-CTG CTG CTG GAA TTC TGT AAA ACC GCC AAT GGT ACC GCT-3′(SEQ ID No. 11)) and SM127 REV (5′-C TGC TGC TGG ATC CGA TCC GCC ACC GCC AGA GCC ACC TCC GCC TGA ACC GCC TCC ACC TGA TTT GTC GTC GTC GTC TTT GTA GTC TCC AGT GGG CAC CAC CAC ATC ATT ATT G-3′(SEQ ID No. 12)), containing EcoRI and BamHI restriction sites, respectively (underlined). Reverse SM127 primer added to FimH sequence FLAG peptide to facilitate monitoring of the displayed recombinant protein and (SGGGG)₃S (SEQ ID No. 15) flexible linker just to improve its assembly on the phage surface. The resulting PCR product was gel purified, digested with EcoRI and BamHI, and ligated into the plasmid pc89 (Felici et al., 1991), in turn digested with EcoRI and BamHI. The resulting plasmid pSTM23 was used to transform DH5αF′ electrocompetent cells.

The DNA fragment coding for the shorter domain ΔFimH (45-159 aa of the mature protein) was amplified by using PCR with primer SM150 FOR (5′-CTG CTG CTG GAA TTC CAT AAC GAT TAT CCG GAA ACC ATT AC-3′(SEQ ID No. 13)) containing EcoRI site and SM127 REV. The resulting PCR fragment coding for the ΔFimH domain, FLAG peptide e flexible linker (SGGGG)₃S (SEQ ID No. 15) was cloned into the plasmid pc89 as above, giving a new phagemid pSTM27. The pSTM27 was used to transform DH5αF′ electrocompetent cells.

### Phage ELISA

Phage ELISA was performed with recombinant phages to confirm the presence of FimH domain or FLAG peptide on the phage surface. ELISA with phage supernatant was performed as follows. Multiwell plates (Immunoplate Maxisorb, Nunc, Roskilde, Denmark) were coated ON at 4° C. with 200 μl of the monoclonal antibody to pill (57D1) (Dente et al., 1994) at concentration of 1 μg/mL in 50 mM NaHCO₃, pH 9.6. After the coating solution had been discarded, the plates were blocked with blocking buffer (5% non-fat dry milk in PBS, 0.05% Tween-20). Phage supernatant diluted with blocking buffer was added to each well and allowed to bind for 1 h at 37° C. A mixture of rabbit anti-FimH serum, diluted 1:50 in blocking buffer and containing 5×10¹⁰ PFU/ml phage fl and 100 μl/ml of HB101 bacterial extract was preincubated for 30 min at 37° C. with slow agitation. The phage solution was discarded, the plates were washed with washing buffer and 200 μl of the serum mixture was added to each well and incubated 60 min at 37° C. The plates were washed and incubated with a 1:5000 dilution of an anti-rabbit HRP-conjugated secondary antibody (A1949, Sigma, St Louis, Mo., USA) for 30 min. Then, the plates were washed and peroxidase activity was detected by incubation with 200 μl TMB liquid substrate system (Sigma). After 15 min of development, the reaction was stopped by adding 25 μl 2M H₂SO₄. The plates were read by an automated ELISA reader (Labsystems Multiskan Bichromatic, Helsinki, Finland) and the results were expressed as A=A₄₅₀ₙₘ−A₆₂₀ₙₘ.

### Phage Propagation and Purification in CsCl Gradient

Single colony of DH5αF′ infected with pSTM27 phage was incubated in 40 mL of LB containing 100 μg/ml ampicillin and 1% glucose until OD=0.2. The bacteria were collected by centrifuging and resuspended in 40 mL of LB with ampicillin without glucose. About 6×10⁹ PFU (plaque-forming units) of helper M13K07 were added to each mL of cell suspension, incubated for 10 min at 37° C. without agitation and for another two h in a shaker. Kanamycin was added to obtain a final concentration of 20 μg/mL, and cells were incubated overnight at 32° C. Phage was purified according to standard PEG/NaCl precipitation (Sambrook et al., 1989) and purified in CsCl gradient as described by Marvin in 1966.

### Hemagglutination Assay

Hemagglutination assay was performed as described earlier (Clark and Bavoil, 1994). Briefly, bacterial culture was grown in LB at 37° C. for 48 h without agitation. The bacterial cells were spun down, washed once with 1×PBS and then resuspended in PBS to get density about 1×10¹⁰ bacteria/ml (cells were concentrated 7-10 times). A guinea pig was anaesthetized with isoflurane, by using Ohmeda Isotec 5 (BOC Healthcare). Guinea pig's blood was collected by cardiac puncture. RBC (red blood cells) suspension was obtained by adding to guinea pig blood a ⅕ of the 20 mM EDTA solution and by diluting the blood 1:4 with PBS or with 1% D-mannose in PBS. A drop of bacterial suspension (40 μl) was mixed with RBC suspension (40 μl) in ELISA plate, after 1-2 min rotation the plate was incubated at RT for 10 min. Degree of HA was estimated in the presence or absence of mannose.

### Immunization

Immunization of mice was performed as described by Poggio et al., 2006. For i.m. immunization a group of 6-week old BALB/c female mice was immunized with a recombinant bacteriophage pSTM27 alone in dose 10¹⁰ PFU (phage forming units), in combination with CFA (Complete Freund's Adjuvant) (in ration 1:1), in combination with CpG oligonucleotide (5′-TCCATGACGTTCCTGACGTT-3′ (SEQ ID No. 16)) or in combination with non CpG oligonucleotide (TCCATGAGCTTCCTGACGTT (SEQ ID No. 17)). The CpG oligonucleotide, described previously (McCluskie & Davis, 1998), was used at 10 μg/dose. Mice after primary immunization were boosted on day 28 post-vaccination. The antigen was delivered to each mouse in a final volume of 100 μL/dose.

For mucosal i.n (intranasal) immunizations, group of mice was immunized with the recombinant bacteriophage pSTM27, under general anesthesia. The phage suspension was instilled to each mouse by contact of a drop to nose. Mice were immunized with 10¹⁰ PFU of pSTM27/dose. After primary immunization mice were boosted on days 7, 14, and 28 post-vaccination. The antigen was delivered in a volume of 7 μl in each nostril in the case i.n. vaccination.

Challenge of the Immunized Mice with the UPEC Strain PT27

Challenge of mice with E. coli strains was essentially performed as described by Hopkins et al., (1995). Strain of type 1 piliated bacteria (E. coli UPEC PT27) was grown in static broth to induce type I pili expression for 48 hrs. Expression of FimH protein was confirmed by HA test in vitro. One-two day before surgery urine was collected from every mice after gentle massage of the abdomen and tested for sterility on LA (Luria Broth agar) plates. Female 6- to 8-week old mice (Harlan) were anesthetized with 0.25 mL/mouse of 2.5% 2,2,2-tribromoethenol, 2.5% 2-methyl-2-butanol in physiologic solution. The bladder of each mouse will be empted by gentle massage of the abdomen. The bacterial suspension, adjusted to contain 10⁸ CFU in 10 μL, was instilled into bladder through urethra to a depth of 1 cm by using a syringe fitted with 0.61 mm outside-diameter silicon catheter (SIL-C20, Solomon Scientific, San Antonio, Tex.). On the fifth day after bacterial infection the mice were sacrificed, the bladder was removed, washed and homogenized in 1 mL of sterile PBS. Different dilution of this homogenate were grown and counted on LA plates.

### Sample Collection

On day 45 the blood from mice were sampled by saphenous vein puncture. Vaginal secretions were collected as described previously (Parr et al., 1998). Mouse vagina was washed with 3 portions of PBS, 50 μl each. The washes were centrifuged immediately after collection and stored at −20° C. The vaginal washes were tested diluted 1:1 in blocking buffer.

### ELISA

Detection of FimH specific IgG and IgA antibodies in sera and vaginal washes of the immunized animals was performed as following. Each well was coated ON with 5 μg/ml of FimH in bicarbonate buffer and blocked. Twofold serial dilution of mice sera starting from 1/200 or 1/1 diluted vaginal washes were added to each well and allowed to bind for 1 h at 37° C. The plates were washed with washing buffer and the FimH-specific mouse IgG and IgA were detected with HRP-conjugated anti-mouse IgG (A9309, Sigma) and anti-mouse IgA (A4789, Sigma) secondary antibodies. The plates were developed and read as above.

### Results

### Expression and Purification of the Recombinant FimH Domain

The FimH domain (3-158 aa of the mature protein) was cloned in pKM16 plasmid (Pavoni et al., 2007) generally used for production of soluble antibodies in scFv configuration. This plasmid directs protein expression under the control of the lacP promoter, allowing expression of the cloned protein fused to the leader peptide of alkaline phosphatase, at its amino terminus, and to 6His-tail at the carboxy terminus. The resulting plasmid pSTM21 was used to transform DH5αF′ cells. We analyzed freeze-thaw purified periplasmic proteins and cell pellet in Western blot developed with an anti-FLAG secondary antibody. Unexpectedly, we found the FimH was not secreted in periplasmic space and we observed anti-FLAG-stained band in the cell pellet (data not shown). To purify FimH protein from cytoplasm we used protocol for purification of insoluble cytoplasmic proteins as in Materials and methods. The purity of the protein was analyzed by SDS-PAGE and Western blot developed with an anti-FLAG secondary antibody. Many additional bands were seen on the gel stained with Coomassie Brilliant Blue 8250. Thus the amount of the FimH protein marked with FLAG was only about 10% of the total protein. The FimH protein was used for rabbit immunization. The polyclonal anti-FimH serum with antibody titer about 1/70000 was obtained.

Display of the FimH Domain as pVIII Protein Fusion

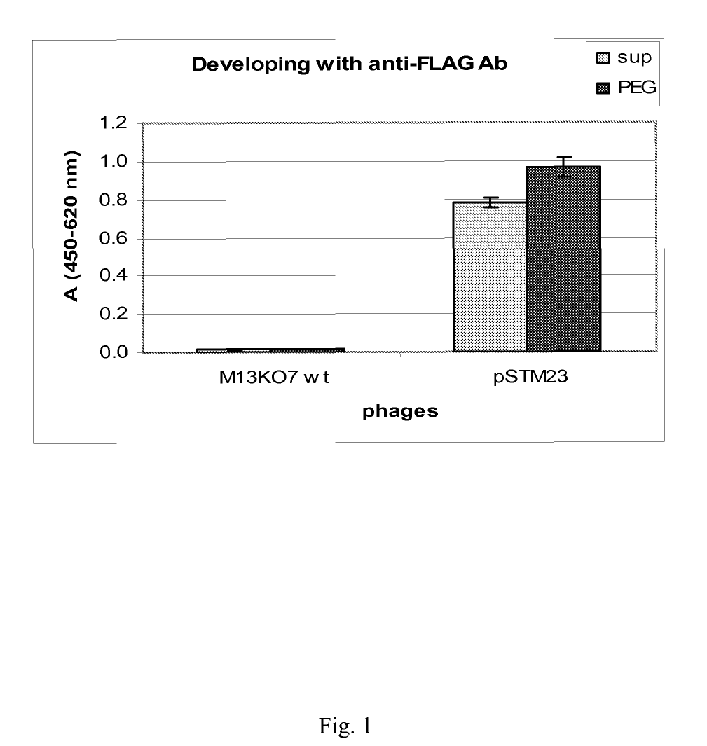

We constructed the pSTM23 plasmid coding for the FimH lectin domain (3-158 aa of the mature protein) fused to the major coat protein pVIII and used it to transform DH5αF′ cells. The fused FimH-pVIII gene was designed to encode also for a flexible linker and FLAG peptide to facilitate the assembly of the recombinant protein and for monitoring of the display efficiency. The transformed cells were grown and the pSTM23 phage was amplified and purified as described in Materials and Methods. ELISA test performed with a monoclonal anti-FLAG antibody showed that recombinant protein is assembled on the phage surface and it is accessible for anti-FLAG antibody (FIG. 1).

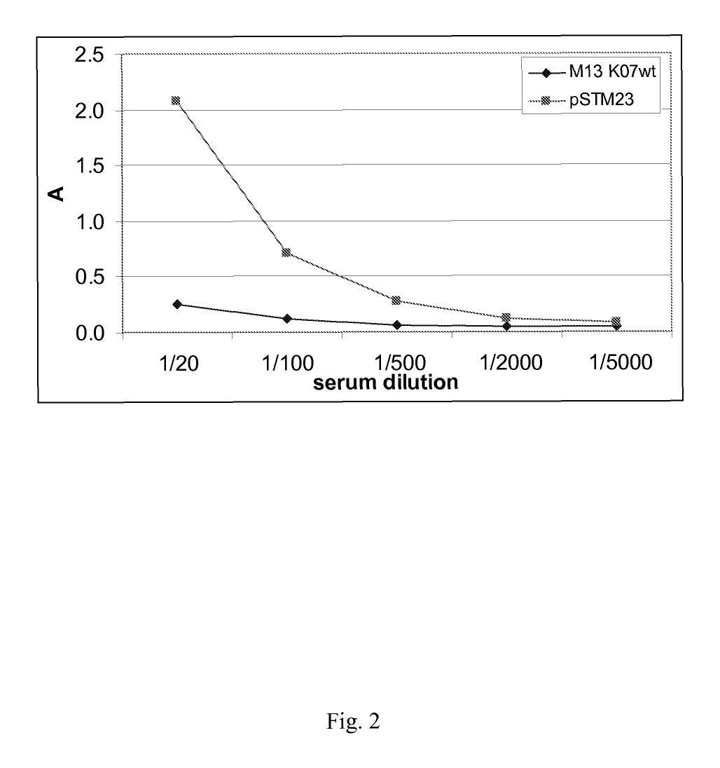

ELISA test performed with anti-FimH polyclonal serum showed specific recognition of FimH displaying phage as compared with wild type phage M13 K07 (FIG. 2).

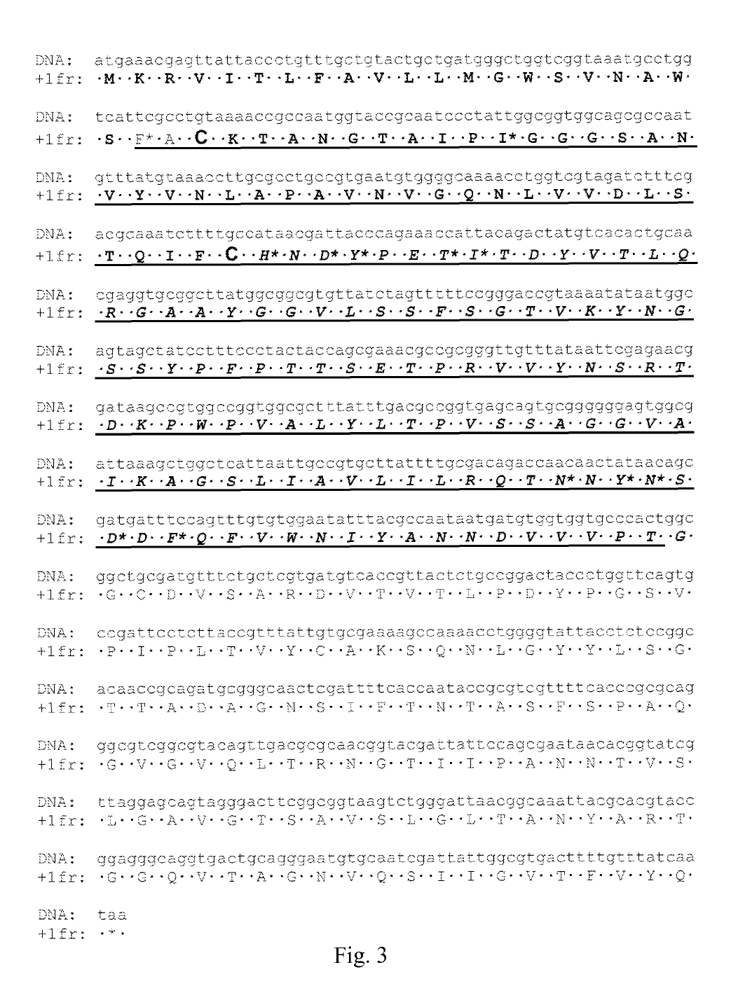

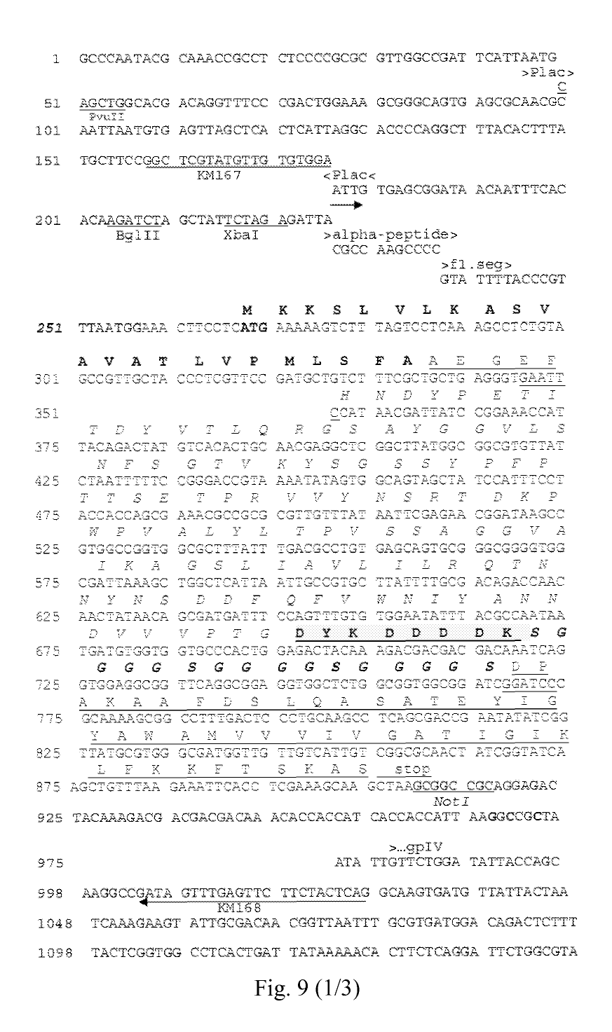

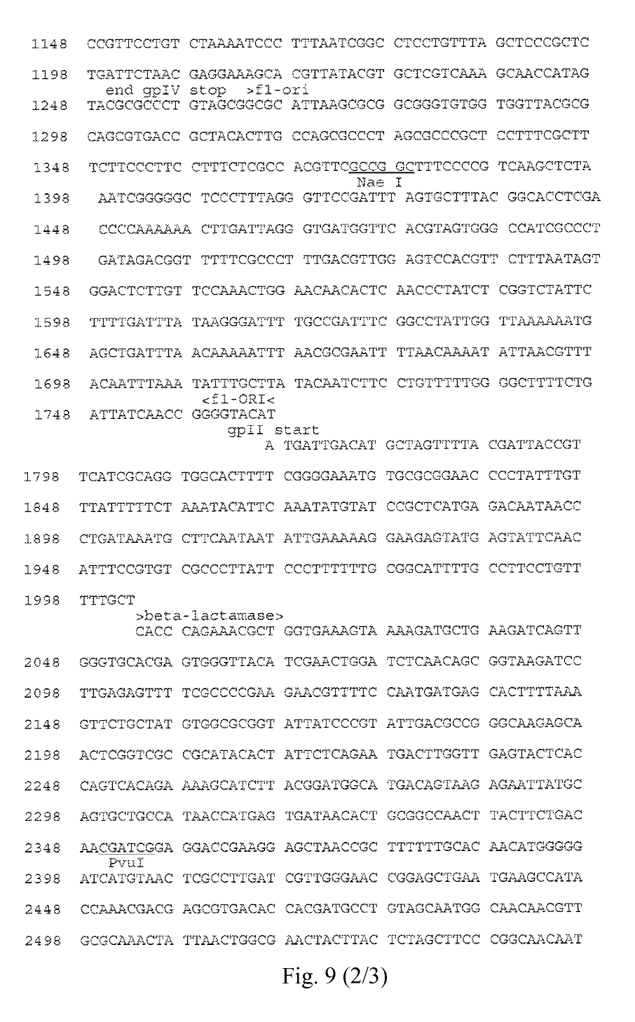

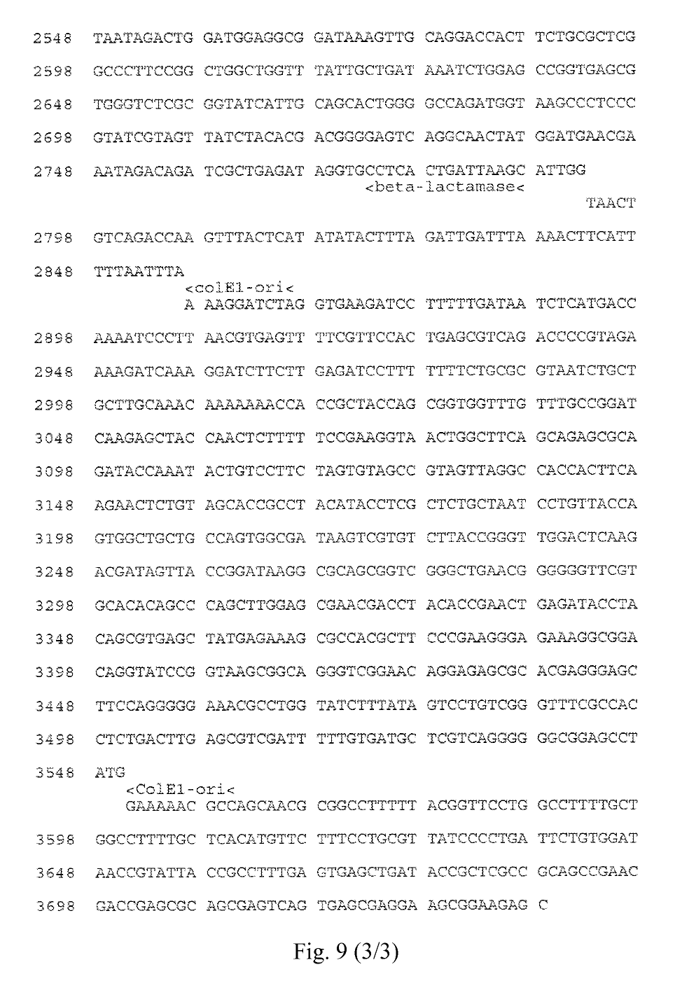

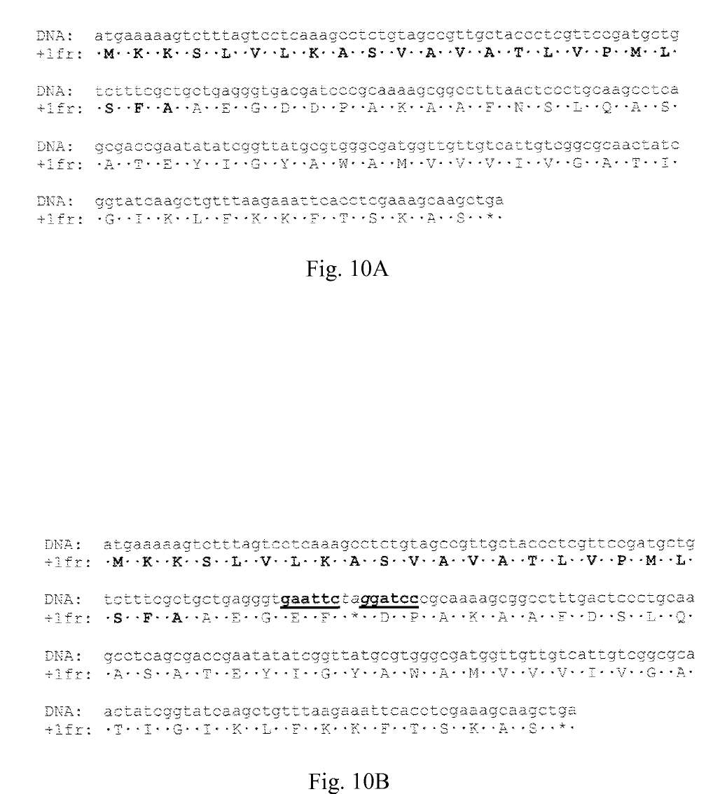

Amino acid sequence analysis of the FimH domain allowed us to assume that the deletion of the fragment 3-44 aa of the mature protein, which includes two cysteine residues and does not contribute significantly to the binding of the mannose (Tchesnokova et al., 2008) could improve phage particle production. We expressed and displayed deleted variant of FimH, 45-159 aa of the mature preotein (FIG. 3). The resulting plasmid pSTM27 (whose sequence is reported in FIG. 9 (SEQ ID No. 3)) coded for ΔFimH protein, FLAG peptide and flexible linker fused to the pVIII. The pSTM27 was used to transform DH5αF′ cells. The transformed cells were grown and the pSTM27 phage was amplified and purified as described in Materials and Methods. ELISA test performed with anti-FimH policlonal serum indicated a significant improvement of phage production for phage pSTM27. The yield of the phage particles for pSTM27 was at least 5 times higher as compare to pSTM23.

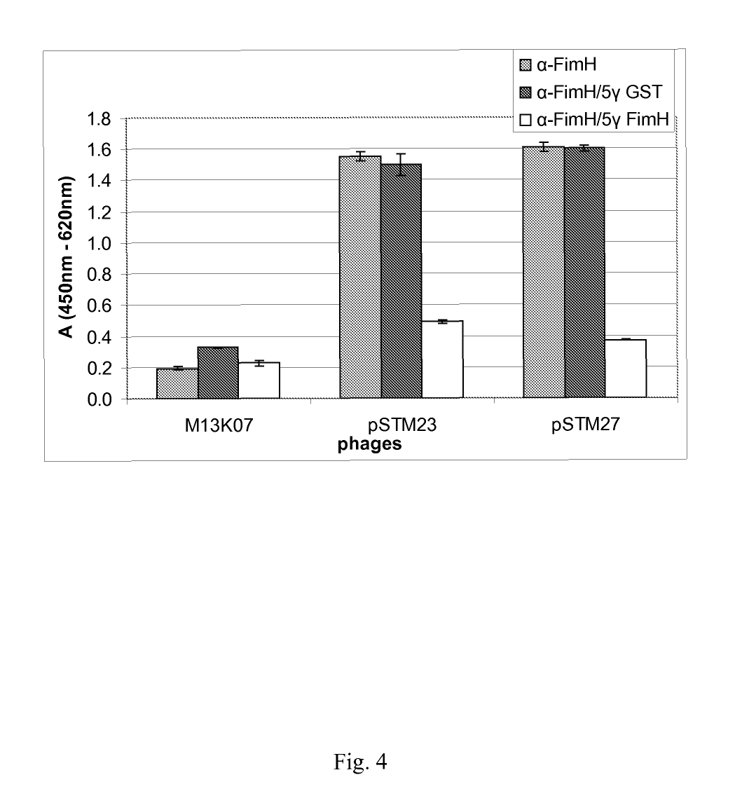

We checked specificity of the recognition of anti-FimH serum to recombinant phages pSTM23 and pSTM27, displaying both a longer and a shorter forms of FimH domain by competing with purified FimH protein. The adding to the reaction mixture of the FimH protein blocked completely phage recognition by serum, thus confirming the specificity of FimH domain binding (FIG. 4).

### Purification of FimH-Displaying Phage

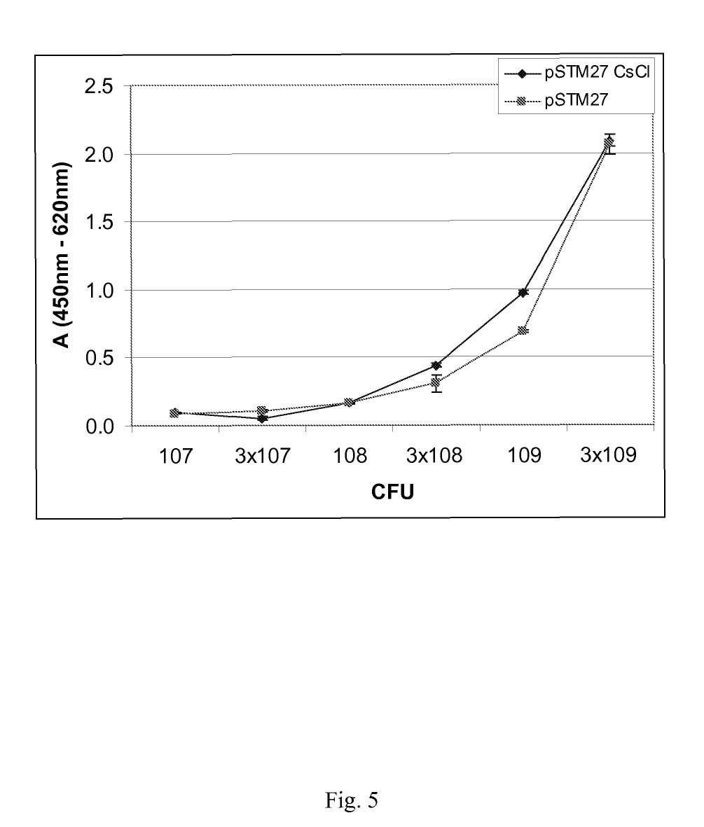

We purified phage particles by standard method of precipitation with PEG/NaCl (Sambrook et al., 1989) and then in CsCl gradient according to the procedure described by Marvin (1966). We analyzed the pSTM27 phage reactivity against anti-FimH serum before and after purification in CsCl gradient to estimate any possible degradation or shedding the FimH domain displayed on the phage particle (FIG. 5). This test demonstrated that pSTM27 phage is stable in the procedure of purification in CsCl gradient and maintains the displayed protein on the phage surface without any lost of specific reactivity.

### Immunization of Mice

Table 1 presents the schedule for immunization, specimen collection and bacterial challenge. Table 2 presents vaccination groups of animals.

TABLE 1
The schedule showing intramuscular (i.m.) and intranasal (i.n.)
immunization, bacterial challenge, specimen collection and sacrifice.

| Intranasal | Intramuscular | Infection/Specimen | |
|---|---|---|---|
| immunization on | immunization on | collection on | Sacrifice on |
| 0 day | 0 day | | |
| 7 day | | | |
| 14 day | | | |
| 28 day | 28 day | | |
| | | 45 day | |
| | | 46 day | |
| | | | 50 day |
| | | | 51 day |

TABLE 2
Six groups of mice were used in immunization routes.

| | Number | Route of | |
|---|---|---|---|
| Group | of mice | immunization | Immunization with |
| 1 | 6 | unimmunized | — |
| 2 | 6 | i.m. | pSTM27 |
| 3 | 6 | i.m. | pSTM27 + CFA |
| 4 | 6 | i.m. | pSTM27 + CpG oligo |
| 5 | 6 | i.m. | pSTM27 + non CpG oligo |
| 6 | 6 | i.n. | pSTM27 |

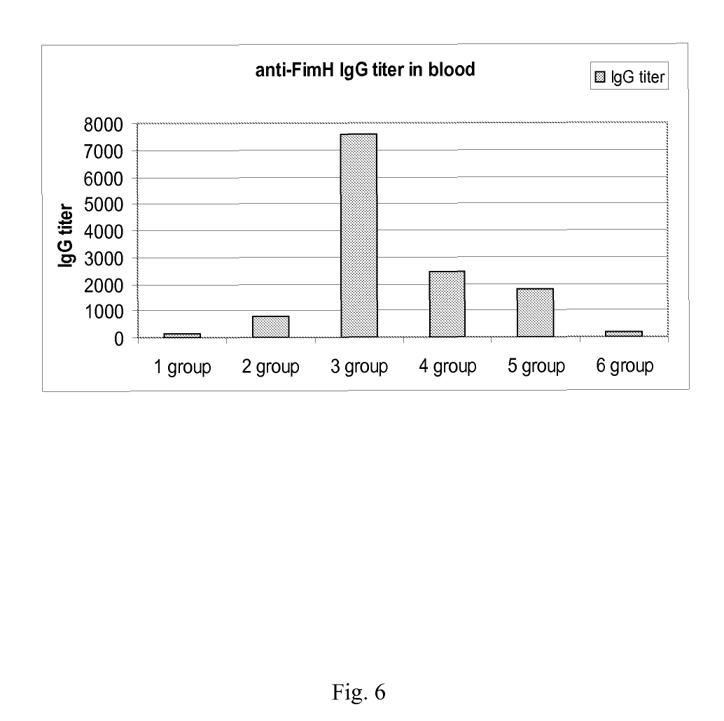

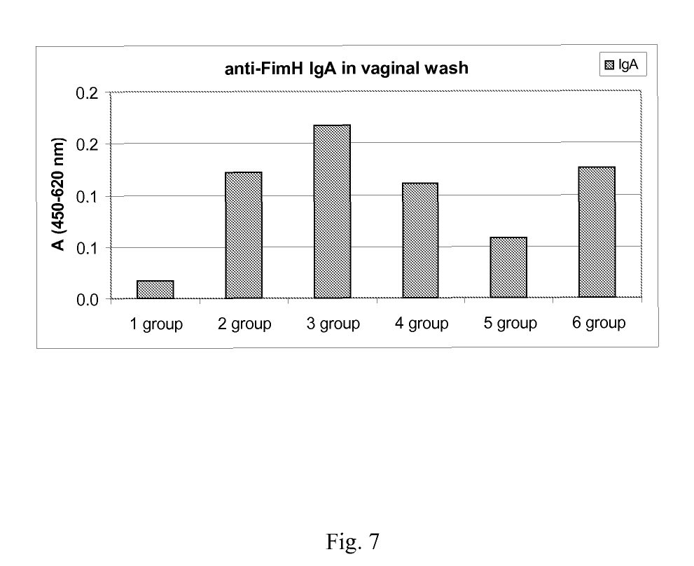

Serum titers of IgG against FimH that were induced in mice by intramucular immunization with either phage pSTM27 alone or in combination with CFA or CpG oligo were significantly higher than those observed in unimmunized mice (FIG. 6) and higher than IgG levels induced by mucosal vaccination. However levels of specific IgA, in vaginal wash of the mice, induced by i.n. or i.m. immunization were comparable (FIG. 7).

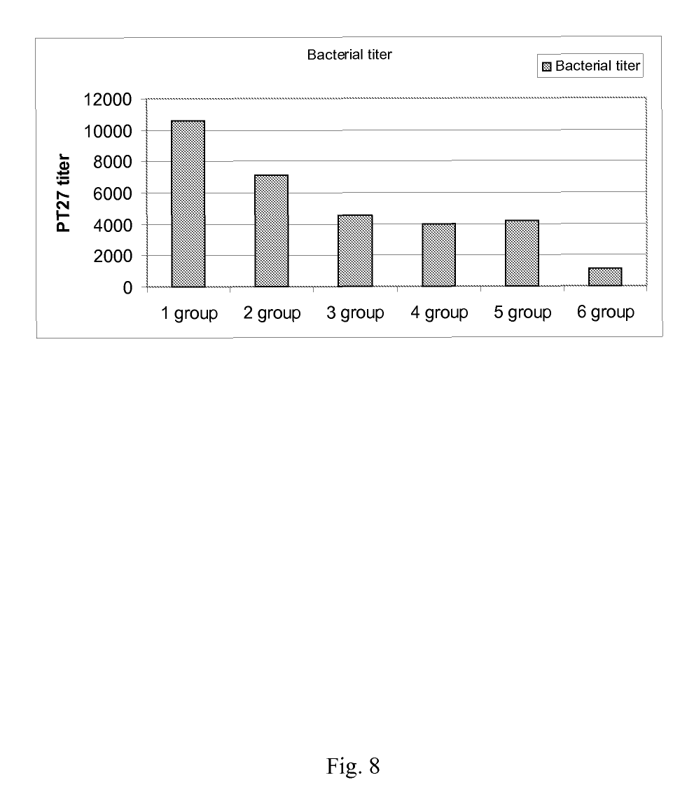

The levels of specific IgA correlated with in vivo protection assay against UPEC clinical strain PT27 (FIG. 8). As it can be seen the mice immunized by mucosal routes, i.e. intranasal, were protected against bacterial infection better than immunized by i.m. routes. Colonization of the bladder was reduced up to 10 times after urethral instillation of UPEC in the case of mucosal vaccinations, as compare to 1.5-2.5-folds for i.m. immunization.

In conclusion, we observed clear positive effect, which consists in reducing in bacterial recovery after UPEC challenge and the reduction correlated with mucosal levels of specific IgA against FimH adhesin domain but not with blood IgG levels.

### REFERENCES

Bearden C M, Agarwal A, Book B K, Vieira C A, Sidner R A, Ochs H D, Young M, Pescovitz M D. Rituximab inhibits the in vivo primary and secondary antibody response to a neoantigen, bacteriophage phiX174. Am J. Transplant. 2005 January; 5(1):50-7.
Bernstein L J, Ochs H D, Wedgwood R J, Rubinstein J. Pediatr. A defective humoral immunity in pediatric acquired immune deficiency syndrome. 1985 September; 107(3):352-7. Clark V L & Bavoil P M. Bacterial pathogenesis in Methods in Enzymology 1994, v.236. 213, Academic Press, Inc.
Clark J R, March J B. Bacterial viruses as human vaccines? Expert Rev Vaccines. 2004 August; 3(4):463-76.
Dente L, Cesareni G, Micheli G, Felici F, Folgori A, Luzzago A, Monaci P, Nicosia A, Delmastro P. Monoclonal antibodies that recognise filamentous phage: tools for phage display technology. Gene. 1994 Oct. 11; 148(1):7-13.
Esposito M, Luccarini I, Cicatiello V, De Falco D, Fiorentini A, Barba P, Casamenti F, Prisco A. Immunogenicity and therapeutic efficacy of phage-displayed beta-amyloid epitopes. Mol. Immunol. 2008; 45(4):1056-62.
Felici F, Castagnoli L, Musacchio A, Jappelli R, Cesareni G. Selection of antibody ligands from a large library of oligopeptides expressed on a multivalent exposition vector. J Mol Biol. 1991 Nov. 20; 222(2):301-10.
Garofalo C K, Hooton T M, Martin S M, Stamm W E, Palermo J J, Gordon J I, Hultgren S J. Escherichia coli from urine of female patients with urinary tract infections is competent for intracellular bacterial community formation. Infect Immun. 2007 January; 75(1):52-60.
Hooton T M, Stamm W E. Diagnosis and treatment of uncomplicated urinary tract infection. Infect Dis Clin North Am. 1997 September; 11(3):551-81.
Hopkins W J, Elkahwaji J, Beierle L M, Leverson G E, Uehling D T. Vaginal mucosal vaccine for recurrent urinary tract infections in women: results of a phase 2 clinical trial. J. Urol. 2007 April; 177(4):1349-53;
Hopkins W J, Hall J A, Conway B P, Uehling D T. Induction of urinary tract infection by intraurethral inoculation with Escherichia coli: refining the murine model. J Infect Dis. 1995 February; 171(2):462-5.
Langermann S, Palaszynski S, Barnhart M, Auguste G, Pinkner J S, Burlein J, Barren P, Koenig S, Leath S, Jones C H, Hultgren S J. Prevention of mucosal Escherichia coli infection by FimH-adhesin-based systemic vaccination. Science. 1997; 276(5312):607-11.
Lidqvist M, Nilsson O, Holmgren J, Hall C, Fermér C. Phage display for site-specific immunization and characterization of high-risk human papillomavirus specific E7 monoclonal antibodies. J Immunol Methods. 2008; 337(2):88-96
Marvin D A. X-ray diffraction and electron microscope studies on the structure of the small filamentous bacteriophage fd. J Mol. Biol. 1966 January; 15(1):8-17.
McCluskie M J, Davis H L. CpG DNA is a potent enhancer of systemic and mucosal immune responses against hepatitis B surface antigen with intranasal administration to mice. J. Immunol. 1998 Nov. 1; 161(9):4463-6.
Minenkova O P, Ilyichev A A, Kishchenko G P, Petrenko V A. Design of specific immunogens using filamentous phage as the carrier. Gene. 1993; 128(1):85-8.
Mysorekar I U, Hultgren S J Mechanisms of uropathogenic Escherichia coli persistence and eradication from the urinary tract. Proc Natl Acad Sci USA. 2006 Sep. 19; 103(38):14170-5.
Naber K G, Cho Y H, Matsumoto T, Schaeffer A J. Immunoactive prophylaxis of recurrent urinary tract infections: a meta-analysis. Int J Antimicrob Agents. 2009 February; 33(2):111-9.
Ochs H D, Davis S D, Wedgwood R J. Immunologic responses to bacteriophage phi-X 174 in immunodeficiency diseases. J Clin Invest. 1971 December; 50(12):2559-68.
Pam D. Vaginal vaccine for recorrent urinary-tract infections. Lancet Infect Dis 2002; 2:68.
Parr E L, Bozzola J J, Parr M B. Immunity to vaginal infection by herpes simplex virus type 2 in adult mice: characterization of the immunoglobulins in vaginal mucus. J Reprod Immunol. 1998; 38(1):15-30.
Pavoni E, Monteriù G, Cianfriglia M, Minenkova O. New display vector reduces biological bias for expression of antibodies in E. coli. Gene. 2007 Apr. 15; 391(1-2):120-9.
Poggio T V, La Torre J L, Scodeller E A. Intranasal immunization with a recombinant truncated FimH adhesin adjuvanted with CpG oligodeoxynucleotides protects mice against uropathogenic Escherichia coli challenge. Can J. Microbiol. 2006; 52(11):1093-102. Reigstad C S, Hultgren S J, Gordon J I. Functional genomic studies of uropathogenic Escherichia coli and host urothelial cells when intracellular bacterial communities are assembled. J Biol. Chem. 2007 Jul. 20; 282(29):21259-67.
Sambrook J, Fritsch E F, Maniatis T: Molecular cloning: A laboratory manual. Volume 2. 2nd edition. Cold Spring Harbor: Cold Spring Harbor Laboratory Press; 1989:4.29-4.30.
Schulman C C, Corbusier A, Michiels H, Taenzer H J. Oral immunotherapy of recurrent urinary tract infections: a double-blind placebo-controlled multicenter study. J. Urol. 1993 September; 150(3):917-21.
Stamm W E. Urinary tract infections. Infect Dis Clin North Am. 2003 June; 17(2):xiii-xiv. Tchesnokova V, Aprikian P, Yakovenko O, Larock C, Kidd B, Vogel V, Thomas W, Sokurenko E. Integrin-like allosteric properties of the catch bond-forming FimH adhesin of Escherichia coli. J Biol. Chem. 2008 Mar. 21; 283(12):7823-33. Epub 2008 Jan. 3.
Thankavel K, Madison B, Ikeda T, Malaviya R, Shah A H, Arumugam P M, Abraham S N. Localization of a domain in the FimH adhesin of Escherichia coli type 1 fimbriae capable of receptor recognition and use of a domain-specific antibody to confer protection against experimental urinary tract infection. J Clin Invest. 1997 Sep. 1; 100(5):1123-36.
Vieira J, Messing J. Production of single-stranded plasmid DNA. Methods Enzymol. 1987; 153:3-11.
Wedgwood R J, Ochs H D, Davis S D. The recognition and classification of immunodeficiency diseases with bacteriophage phiChi 174. Birth Defects Orig Artic Ser. 1975; 11(1):331-8.
Willis A E, Perham R N, Wraith D. Immunological properties of foreign peptides in multiple display on a filamentous bacteriophage. Gene. 1993; 128(1):79-83.

1. A recombinant bacteriophage displaying at its surface multiple copies of a chimeric polypeptide, said chimeric polypeptide comprising at least:
a) a coat protein of a filamentous bacteriophage or a functional fragment thereof able to be expressed on the surface of the bacteriophage and
b) an immunogenic domain of bacterial adhesion protein FimH consisting of SEQ ID No. 2.

2. The recombinant bacteriophage according to claim 1, wherein the filamentous bacteriophage belongs to the group of M13, fd and fl.

3. The recombinant bacteriophage according to claim 1, wherein the coat protein is the coat protein p VIII.

4. The recombinant bacteriophage according to claim 1, wherein the immunogenic domain of the bacterial adhesion protein FimH is a lectin-binding domain.

5. The recombinant bacteriophage according to claim 4, wherein the domain is fragment 24-179 aa of SEQ ID No. 2.

6. The recombinant bacteriophage according to claim 4, wherein the domain is fragment 66-180 aa of SEQ ID No. 2.

7. The recombinant bacteriophage according to claim 1, wherein the chimeric polypeptide further comprises a linker between the coat protein or fragment thereof and the immunogenic domain of the bacterial adhesion protein FimH.

8. The recombinant bacteriophage according to claim 1, wherein the chimeric polypeptide further comprises an affinity tag.

9. The recombinant bacteriophage according to claim 1, having the nucleic acid sequence essentially consisting of SEQ ID No. 3.

10. A medicament comprising the recombinant bacteriophage according to claim 1.

11. The medicament according to claim 10, wherein the recombinant bacteriophage is comprised in an effective amount for use as an immunogen against urinary tract infections (UTI).

12. A pharmaceutical composition comprising the recombinant bacteriophage according to claim 1, in association with a physiologically-acceptable carrier or diluent.

13. The pharmaceutical composition according to claim 12, wherein the recombinant bacteriophage is formulated to elicit a humoral and/or cellular immune response.

14. The pharmaceutical composition according to claim 13, wherein the recombinant bacteriophage is formulated for use in vaccinating against urinary tract infections (UTI).

15. The pharmaceutical composition according to claim 13 further comprising an adjuvant.

16. The pharmaceutical composition according to claim 13 wherein the recombinant bacteriophage is associated with a vehicle.

17. The pharmaceutical composition according to claim 12, wherein the recombinant bacteriophage is formulated for use in prophylaxis and/or treatment of urinary tract infections (UTI) in a human or animal.

18. A method to treat a patient, the method comprising
administering to the patient the recombinant bacteriophage according to claim 1 in an effective to treat or prevent a condition associated with bacterial adhesion protein FimH.

19. The method of claim 18, wherein the condition is urinary tract infections (UTI) and the administering is performed to elicit an immunogenic response against urinary tract infections (UTI).

20. The method of claim 18, wherein the administering is performed to vaccinate the patient against urinary tract infections (UTI).
